(19) United States
(12) Patent Application Publication
(10) Pub. No.: US 2015/0344841 A1
(43) Pub. Date: Dec. 3, 2015

(54) LACTOBACILLUS HAVING ABILITY TO INDUCE IL-12 PRODUCTION, AND METHOD FOR CULTURING SAME

(71) Applicants: BIOGENICS KOREA CO., LTD., Dongdaemun-gu, Seoul (KR); BIO-LAB LTD., Saitama (JP); THE JAPANESE ASSOCIATION OF CLINICAL RESEARCH ON SUPPLEMENTS, Saitama (JP)

(72) Inventors: Tatsuhiko KAN, Sayama-shi (JP); Makoto OHWAKI, Hidaka-shi (JP)

(73) Assignee: BIOGENICS KOREA cO., lTD., SEOUL (KR)

(21) Appl. No.: 14/650,341

(22) PCT Filed: Jul. 22, 2013
(86) PCT No.: PCT/KR2013/006528

§ 371 Date: Jun. 8, 2015

(30) Foreign Application Priority Data
Dec. 7, 2012 (JP) 2012-267793

(51) Int. Cl. C12N 1/38; C12N 1/20

(52) U.S. Cl. CPC C12N 1/38; C12N 1/20

(57) ABSTRACT

A method of producing a large volume of Lactobacillus bacteria and increasing the activity of the obtained Lactobacillus is provided. The Lactobacillus is prepared by culturing in a culture medium and adding an alkaline chemical while controlling the pH, and sterilizing the Lactobacillus by applying stress in the later stage of the cultivation so as to produce Lactobacillus having the ability to induce IL-12 production. Applying stress is selected from the group consisting of: (a) cultivating without adding an alkaline chemical; (b) cultivating at a temperature range in which propagation is suppressed; (c) cultivating by adding 1% by weight or more of salt; and/or (d) cultivating at pH of 5 or below.

### TECHNICAL FIELD

This invention relates to Lactobacillus having an ability to induce IL-12 production, and a method for preparing the same.

### BACKGROUND ART

Lactobacillus sp. is known to have an effect of relieving allergy symptoms or improving immune activities in addition to improvement of intestinal flora. For example, IL-12 is a cytokine which is secreted from antigen-presenting cells such as dendritic cells or macrophages. In this case, IL-12 is a very potent immunostimulator that activates natural killer cells (NK cells), lymphokine-activated killer cells (LAK cells) or killer T cells (CTL cells) which directly attack cancer cells, or enhances production of interferon-γ (IFN-γ). Also, IL-12 has an immunoregulatory function of shifting the Th1/Th2 balance into Th1. Lactobacillus sp. is known to have the ability to induce IL-12 production (Patent Document 1). When the Lactobacillus sp. is orally ingested, it can directly act on immunocompetent cells taking part in gut immunity to induce the IL-12 production, thereby promoting immunostimulation or mitigation of allergy symptoms.

Meanwhile, a variety of culture mediums are used to culture Lactobacillus sp. In this case, culture mediums having rich nutrients, including a yeast extract, peptone, a meat extract, and the like, have been generally used to facilitate the growth of Lactobacillus sp. However, the propagation of the Lactobacillus sp. is inhibited since pH of the culture medium is reduced due to its metabolites (for example, lactic acid, etc.). Therefore, a neutralization culture in which the pH of the culture medium is adjusted in the vicinity of neutral pH is performed.

Also, the culture medium conditions are known to have an influence on the activities of the Lactobacillus sp. For example, Patent Document 2 discloses that a Lactobacillus strain having a high immunostimulatory effect is obtained by culturing Lactobacillus sp. using a culture medium containing corn steep liquor and a hydrolysate of casein. Further, Patent Document 3 discloses that a Lactobacillus strain having a high immunoregulatory effect is obtained by culturing Lactobacillus sp. in a culture medium (pH 3.5 to 5.0).

### PRIOR-ART DOCUMENTS

### Patent Documents

Patent Document 1: Japanese Patent No. 4621218

Patent Document 2: Japanese Patent Laid-open Publication No. 2004-41099

Patent Document 3: International Publication No. 2007/138993

### SUMMARY OF INVENTION

### Technical Problem

However, technology of changing the culture medium conditions to enhance the activities of Lactobacillus sp. as disclosed in Patent Documents 2 and 3 is rather unfavorable in an aspect of reducing preparation costs so that Lactobacillus sp. is cultured to obtain a large volume of the Lactobacillus strain, and thus there is no conventional technology compatible to the technology.

Therefore, it is an aspect of the present invention to provide novel technology capable of preparing a large volume of a Lactobacillus strain and also enhancing the activity of the Lactobacillus strain.

### Solution to Problem

The present inventors have conducted research to solve the above problems, and found that, when a neutralization culture is performed to realize a large volume of Lactobacillus sp. to prepare a Lactobacillus strain, the activity of the Lactobacillus sp. is reduced compared to when the Lactobacillus sp. is cultured without adjusting pH, but the activity of the Lactobacillus sp. may be recovered or further enhanced by keeping the Lactobacillus sp. in an environment which is undesirable to the growth of the Lactobacillus sp. at a later stage of the cultivation after the neutralization culture. Therefore, the present invention has been completed based on these facts.

That is, the present invention provides a Lactobacillus strain having an ability to induce IL-12 production, and a method for preparing the same, as configured as follows.

[1] A method for preparing a Lactobacillus strain having an ability to induce IL-12 production, the method characterized in that it includes adding an alkaline chemical to a culture medium to adjust pH, cultivating Lactobacillus sp. in the culture medium, and sterilizing the Lactobacillus sp. by applying stress at a later stage of the cultivation.

[2] The method of paragraph [1], wherein the stress is at least one selected from the group consisting of (a) cultivating the Lactobacillus sp. without adding the alkaline chemical; (b) cultivating the Lactobacillus sp. in a temperature range in which the propagation of the Lactobacillus sp. is suppressed; (c) cultivating the Lactobacillus sp. after a salt is added at a concentration of 1% by weight or more in a culture broth; and (d) cultivating the Lactobacillus sp. at pH 5 or less.

[3] The method of paragraph [1] or [2], wherein the Lactobacillus sp. is sterilized in the culture broth after the cultivation, and then collected.

[4] The method of any one of paragraphs [1] to [3], wherein the Lactobacillus sp. is isolated from a plant.

[5] The method of any one of paragraphs [1] to [4], wherein the Lactobacillus sp. is a microorganism belonging to Lactobacillus plantarum, Lactococcus lactis sp. cremoris, Enterococcus faecalis, or Lactobacillus brevis.

[6] The method of any one of paragraphs [1] to [5], wherein the sterilizing of the Lactobacillus sp. is performed at 80° C. or higher.

[7] The method of paragraph [3], wherein the corresponding sterilization is performed so that a brightness L* value is less than or equal to 65 and a chromaticity a* value is greater than or equal to −4, the brightness L* and chromaticity a* values being obtained when the Lactobacillus sp. is sterilized in the culture broth after the cultivation, the culture medium is removed by subjecting the culture broth to centrifugation, membrane separation, or precipitation, 5 g of the resulting strain concentrate having a solid content of 4 to 7% by weight is added into a quartz glass Petri dish having a diameter of 35 mm, and a colorimetric color of the L*a*b* color system is measured for the strain concentrate per se.

[8] A Lactobacillus strain having an ability to induce IL-12 production, characterized in that it is obtained by adding an alkaline chemical to a culture medium to adjust pH, cultivating Lactobacillus sp. in the culture medium, and sterilizing the Lactobacillus sp. by applying stress at a later stage of the cultivation.

[9] The Lactobacillus strain of paragraph [8], which is obtained by sterilizing the Lactobacillus sp. in a culture broth after the cultivation, followed by collecting the Lactobacillus sp.

[10] The Lactobacillus strain of paragraph [8] or [9], which is obtained by sterilizing of the Lactobacillus sp. at 80° C. or higher.

[11] The Lactobacillus strain of paragraph [9], which is obtained by performing the corresponding sterilization so that a brightness L* value is less than or equal to 65 and a chromaticity a* value is greater than or equal to −4, the brightness L* and chromaticity a* values being obtained when the Lactobacillus sp. is sterilized in the culture broth after the cultivation, the culture medium is removed by subjecting the culture broth to centrifugation, membrane separation, or precipitation, 5 g of the resulting strain concentrate having a solid content of 4 to 7% by weight is added into a quartz glass Petri dish having a diameter of 35 mm, and a colorimetric color of the L*a*b* color system is measured for the strain concentrate per se.

### Advantageous Effects of Invention

According to one exemplary embodiment of the present invention, the activity of Lactobacillus sp. reduced due to a neutralization culture, especially, an ability to induce IL-12 production can be recovered or further enhanced by performing a neutralization culture on the Lactobacillus sp. to enhance a growth ability of the Lactobacillus sp. so as to obtain a large volume of a Lactobacillus strain, and keeping the Lactobacillus sp. in an environment which is undesirable to the growth of the Lactobacillus sp. at a later stage of the cultivation after the neutralization culture. Therefore, it is possible to reduce the preparation cost and also prepare a Lactobacillus strain having high activities.

### BRIEF DESCRIPTION OF DRAWINGS

FIG. 1 is an image obtained by comparing staining states of a Lactobacillus strain, which is not sterilized after culturing the Lactobacillus strain, and staining states of a Lactobacillus strain, which is obtained by sterilizing the Lactobacillus strain under a predetermined condition and collecting the Lactobacillus strain.

### DETAILED DESCRIPTION OF EMBODIMENT

Lactobacillus sp. used in the present invention is not particularly limited as long as it has an ability to induce IL-12 production. For example, the Lactobacillus sp. may include microorganisms belonging to the genus Enterococcus such as E. faecalis, and E. faecium, microorganisms belonging to the genus Lactobacillus such as L. acidophilus, L. gasseri, L. mali, L. plantarum, L. buchneri, L. casei, L. johnsonii, L. gallinarum, L. amylovorus, L. brevis, L. rhamnosus, L. kefir, L. paracasei, and L. crispatus, microorganisms belonging to the genus Streptcoccus such as S. thermophilus, microorganisms belonging to the genus Lactococcus such as L. lactis, microorganisms belonging to L. lactis sp. cremoris, microorganisms belonging to the genus Bifidobacterium such as B. bifidum, B. longum, B. adolescentis, B. infantis, B. breve, and B. catenulatum, and the like.

Among the Lactobacillus sp., L. plantarum or L. brevis isolated from plants is particularly preferably applied since the strain has significant synergic effects as will be described later in Examples.

According to one exemplary embodiment of the present invention, Lactobacillus sp. sufficiently grows prior to cultivation to ensure a large volume of a Lactobacillus strain. For this purpose, it is necessary to culture the Lactobacillus strain in a culture medium suitable for propagation of the Lactobacillus strain and simultaneously culture the Lactobacillus strain by adding an alkaline chemical to the culture medium to suppress pH reduction caused by metabolites (for example, lactic acid, etc.) of the Lactobacillus strain and while adjusting a pH value.

An aqueous solution such as sodium hydroxide, potassium hydroxide, or calcium hydroxide, or ammonia may be used as the alkaline chemical. The pH value is preferably maintained in a range of pH 5.8 to 7.5, more preferably pH 6.0 to 6.8. The pH adjustment is manually performed, but may be easily and accurately performed using a pH stat.

The culture medium is not particularly limited as long as it is suitable for the propagation of the Lactobacillus strain. The culture medium suitable for the propagation of the Lactobacillus strain may include culture mediums having rich nutrients, including a yeast extract, peptone, a meat extract, and the like. Commercially available culture mediums that may be used herein may also include a “Difco Lactobacilli MRS broth” (trade name, Becton Dickinson & Company, Japan), “MRS Bouillon MERCK” (trade name, Chemicals Co.), etc. Also, when special compositions of the culture medium are required according to the types of Lactobacillus sp., desired compositions of the culture medium may be used herein, but the present invention is not particularly limited.

As one typical example of the neutralization culture, when it is assumed that E. faecalis is cultured at a starting strain concentration of 1×10⁶ to 1×10⁷ cfu/mL, E. faecalis may propagate in a culture medium of “Difco Lactobacilli MRS broth” (trade name, Becton Dickinson & Company, Japan) until the final strain concentration reaches 5×10⁹ to 5×10¹⁰ cfu/mL when the strain is cultured at 35 to 38° C. for 16 to 30 hours while maintaining a pH value in a range of pH 6.5 to 6.8.

Also, when it is assumed that L. plantarum is cultured at a starting strain concentration of 1×10⁶ to 1×10⁷ cfu/mL, L. plantarum may propagate in a culture medium of “Difco Lactobacilli MRS broth” (trade name, Becton Dickinson & Company, Japan) until the final strain concentration reaches 5×10⁹ to 5×10¹⁰ cfu/mL when the strain is cultured at 30 to 35° C. for 18 to 40 hours while maintaining a pH value in a range of pH 6.0 to 6.2.

According to one exemplary embodiment of the present invention, it is necessary to apply stress to the strain at a later stage of the cultivation after the neutralization culture. Here, the term “stress” generally refers to all environments which are unfavorable to the growth of Lactobacillus sp. Therefore, the activity of the Lactobacillus sp. reduced by the above-described neutralization culture, especially an ability to induce IL-12 production may be recovered, or further enhanced.

A method of applying stress may, for example, include the following methods:

(a) cultivating the Lactobacillus sp. without adding the alkaline chemical;

(b) cultivating the Lactobacillus sp. in a temperature range in which the propagation of the Lactobacillus sp. is suppressed;

(c) cultivating the Lactobacillus sp. after a salt is added at a concentration of 1% by weight or more in a culture broth; and

(d) cultivating the Lactobacillus sp. at pH 5 or less.

In operation (a), the Lactobacillus sp. is kept under low pH conditions as the pH of the culture medium is reduced by lactic acid produced by the Lactobacillus sp., resulting in an environment which is unfavorable for their growth. In this case, it is preferred to culture the Lactobacillus sp. until the pH value reaches at least pH 5 or less without adding the alkaline chemical.

In operation (b), an environment unfavorable to the growth of Lactobacillus sp. is formed by keeping the Lactobacillus sp. at a predetermined temperature. The temperature condition may be properly set to a wide extent according to the Lactobacillus sp. used. For example, the temperature condition is set in a temperature range which is preferably shifted from a temperature range suitable for the growth of Lactobacillus sp. to a high or low temperature of 3 to 12° C., more preferably 5 to 10° C.

In operation (c), an environment unfavorable to the growth of Lactobacillus sp. is formed by keeping the Lactobacillus sp. at a high osmotic pressure. A salt such as NaCl is added so that the salt in the culture broth is preferably included at a concentration of 0.5% by weight or more, more preferably 1 to 2% by weight.

In operation (d), an environment unfavorable to the growth of Lactobacillus sp. is formed by keeping the Lactobacillus sp. at a low pH value. The culture medium is preferably adjusted so that the pH value is less than or equal to be pH 5, more preferably in a range of pH 5 to 3 using an acidulant such as lactic acid.

The method of applying stress is not limited to the above-exemplified methods. For example, stresses caused by other methods may be properly applied. Also, stresses caused by at least two methods may be applied simultaneously or sequentially. In addition, the stresses caused by the same respective methods may be applied at predetermined intervals of time, and stresses caused by other methods may be applied alternately. A mode of applying stress is not particularly limited.

According to one exemplary embodiment of the present invention, it is also necessary to sterilize the Lactobacillus sp. which is completely cultured as described above. Therefore, the self-digestion of the Lactobacillus sp. may be prevented to secure the quality stability.

The sterilization method is not particularly limited, and may include a method of culturing Lactobacillus sp. in the same manner as practiced in the related art, removing a culture medium by means of filtration, centrifugation or precipitation, collecting the Lactobacillus sp., suspending the Lactobacillus sp. in water or a buffer to adjust a concentration of the Lactobacillus concentrate, and autoclaving the Lactobacillus concentrate at 80 to 100° C. Also, even when the Lactobacillus sp. is fed into a spray drying apparatus for the purpose of drying and pulverizing the Lactobacillus sp., the Lactobacillus sp. may be sterilized in this process. Therefore, such a spray drying process may be desirable.

However, as will be described in Examples below, sterilizing the Lactobacillus sp. in a culture broth after the cultivation and collecting the Lactobacillus sp. result in a higher ability to induce IL-12 production, compared to sterilizing the Lactobacillus sp. in a state in which the culture broth is removed after the strain collection. As a result, in one preferred embodiment, the Lactobacillus sp. is sterilized in the culture broth after cultivation, and then collected. In this case, for example, the sterilization method may include a method of heat-sterilizing the Lactobacillus sp. in the culture broth after the cultivation, removing the culture medium by means of filtration, centrifugation or precipitation, and collecting the Lactobacillus sp. Also, a method of removing some of the culture medium by means of filtration, centrifugation or precipitation after the cultivation, preparing a concentrate concentrated 2 to 10 times, subjecting the concentrate to heat sterilization, removing the other culture medium by means of filtration, centrifugation or precipitation, and collecting the Lactobacillus sp. is possible. The heat sterilization is preferably performed at 80° C. or more, more preferably at 80 to 100° C. for 5 to 20 minutes.

However, when the Lactobacillus sp. is sterilized in the culture broth after the cultivation as will be described in Examples below, the resulting Lactobacillus strain is stained with yellowish brown, and an ability to induce IL-12 production is enhanced when the yellowish brown is present at a certain concentration. Therefore, the sterilization is preferably performed so that the yellowish brown of the Lactobacillus strain reaches at least a predetermined concentration. Specifically, the sterilization is preferably performed so that the brightness L* and chromaticity a* values of the L*a*b* color system calculated as follows are less than or equal to 65 and greater than or equal to −4, respectively.

The Lactobacillus sp. is sterilized in the culture broth after the cultivation, the culture medium is removed by subjecting the culture broth to centrifugation, membrane separation, or precipitation, and 5 g of a strain concentrate having a solid content of 4 to 7% by weight is prepared. Then, the brightness L* and chromaticity a* values are calculated when 5 g of the strain concentrate is added into a quartz glass Petri dish having a diameter of 35 mm, and a colorimetric color of the L*a*b* color system is measured for the strain concentrate per se. Also, the colorimetry may be performed using a colorimeter.

The Lactobacillus strain obtained according to one exemplary embodiment of the present invention may be subjected to atomization and reaggregation prevention processes. Therefore, the activity of the Lactobacillus strain, such as an ability of the Lactobacillus strain to induce IL-12 production, and the quality stability may be well maintained. Such a method may include a method disclosed in Japanese Patent No. 4621218. Specifically, the method may be performed as follows.

First, the Lactobacillus strain is pulverized or dispersed until the average particle size of the Lactobacillus strain reaches a nanometer (nm) size less than 1 micrometer (μm), preferably 0.6 μm. These pulverization and dispersion processes may be performed separately or simultaneously. Also, the corresponding particle size may be measured using a particle size distribution meter or an electron microscope to check whether the particle size is less than 1 μm.

An atomization method may include known techniques such as an agitator, a mixer, a ball mill, a bead mill, a jet mill, a homogenizer, a generator, a “nanomizer” (commercially available from Yoshida Machinery Co., Ltd.), an “ultimaizer system” (commercially available from Sugino Machine Co., Ltd.), etc., regardless of wet/dry techniques. In this case, a wet dispersion process is preferably performed on the Lactobacillus strain. For example, the Lactobacillus strain is included at a content of 2 to 30% by weight, more preferably 5 to 20% by weight, based on the calculated dry matter, and the atomization method may be performed by circulating a suspension whose pH value is adjusted to pH 6.0 to 7.0 at a condition of a pressure of 80 to 200 kg/cm², more preferably 100 to 170 kg/kg/cm² using a high-pressure homogenizer.

Next, the reaggregation prevention process is performed using a dispersing agent. Such a method preferably includes adding a dispersing agent to a Lactobacillus strain upon the atomization, atomizing the Lactobacillus strain, and pulverizing the resulting suspension, or atomizing the Lactobacillus strain, adding a dispersing agent to the Lactobacillus strain, and pulverizing the resulting suspension. The pulverization may be performed by spray-drying or freeze-drying the Lactobacillus strain after the atomization. Therefore, the suspension is pulverized in a state in which the dispersing agent is brought into contact with the atomized Lactobacillus strain. As a result, the Lactobacillus strain has good dispersibility even when re-suspended in water, and the activity and quality stability of the Lactobacillus strain may be maintained well since the reaggregation of the atomized Lactobacillus strain is prevented.

Preferred examples of the dispersing agent that may be used herein may include polysaccharides such as dextrin, soluble plant fibers, and indigestible dextrin, low-molecular saccharides such as trehalose, lactose, and maltose, collagen, and peptides such as whey degradation products, and soy protein degradation products. The amount of the added dispersing agent is preferably in a range of 20 to 1,000 parts by weight, more preferably a range of 50 to 1,000 parts by weight, and most preferably a range of 100 to 600 parts by weight, based on 100 parts by weight of the calculated dry matter of the Lactobacillus strain. When the amount of the added dispersing agent is less than 20 parts by weight based on 100 parts by weight of the calculated dry matter of the Lactobacillus strain, an effect of adding the dispersing agent is insufficient. On the other hand, when the amount of the added dispersing agent is greater than 1,000 parts by weight, it is difficult to ensure the content of the Lactobacillus strain. Therefore, both of the two cases are not preferred.

Hereinafter, one use example of the Lactobacillus strain obtained according to one exemplary embodiment of the present invention will be described.

The Lactobacillus strain obtained according to one exemplary embodiment of the present invention has an excellent effect of inducing IL-12 production, and thus is highly suitable as an active ingredient of an IL-12 production inducer, as will be described in Examples below. In this case, the Lactobacillus strain subjected to the atomization and reaggregation prevention processes may be preferably used as it is, or the Lactobacillus strain dried using the spray or freeze drying process may be preferably used. On the other hand, when the atomized Lactobacillus strain is washed with water, components degraded into a low-molecular size by means of the atomization, or nutrient components exposed to the outside by means of the atomization may be washed out. Therefore, both of the two cases are not preferred.

For the IL-12 production inducer, other materials may be blended in addition to the Lactobacillus strain obtained according to one exemplary embodiment of the present invention, but the present invention is not particularly limited thereto. When necessary, a pharmaceutically available base or carrier may be added, and prepared into the form of, for example, a tablet, a granule, a capsule, a pill, a discutient, a solution, a powder, a jelly, and a candy, which may be used as an oral formulation. Also, the pharmaceutically available base or carrier may be prepared into the form of an ointment, a cream a gel, and a lotion, which may be used as a solution for external use on the skin.

For the IL-12 production inducer, a food source may be blended as another material in addition to the Lactobacillus strain obtained according to one exemplary embodiment of the present invention. For example, the food source may include a variety of sugars, an emulsifying agent, a sweetening agent, an acidulant, a fruit juice, a flavor, etc. More particularly, the food source may include saccharides such as glucose, sucrose, fructose, and honey, sugar alcohols such as sorbitol, xylitol, erythritol, lactitol, and palatinit, emulsifying agents such as sucrose fatty acid ester, glycerin fatty acid ester, and lecithin. In addition, a variety of vitamins such as vitamin A, vitamin B, vitamin C, and vitamin E, herbal extracts, grain ingredients, vegetable ingredients, milk ingredients, and the like may also be blended.

For the IL-12 production inducer, the content of the Lactobacillus strain may be properly determined in consideration of the relationship between the amount and the effective dose of the Lactobacillus strain when the IL-12 production inducer is prepared into various forms, but the present invention is not particularly limited. Typically, when the IL-12 production inducer is in a solid form, the IL-12 production inducer is preferably present at a content of 0.1 to 100% by weight, more preferably a content of 1 to 50% by weight, based on a total of the calculated dry matter of the Lactobacillus strain. Also, when the IL-12 production inducer is in a liquid or jelly form, the IL-12 production inducer is preferably present at a content of 0.1 to 50% by weight, more preferably a content of 1 to 5% by weight, based on a total of the calculated dry matter of the Lactobacillus strain.

In the case of the dosage form of the IL-12 production inducer, the IL-12 production inducer may be orally ingested to act in the body, or applied onto the skin. Also, a breathing apparatus may be used to inhale the IL-12 production inducer, but the dosage form is not particularly limited.

When an adult orally ingests the IL-12 production inducer, the dose of the IL-12 production inducer is not particularly limited, but may be typically in a range of approximately 0.01 to 10 g a day when calculated based on the dry matter of the Lactobacillus strain. The shapes or composition of these feed may be determined according to the description of use patterns when the feed is ingested orally.

Meanwhile, the Lactobacillus strain obtained according to one exemplary embodiment of the present invention may be blended with confectionery such cookies, senbei, jelly, sweet bean jelly, yogurt, and Deli Manjoo, foods such as soft drinks, nutritional drinks, and soups, and used. Also, the Lactobacillus strain may be blended with health foods provided for the purpose of maintaining or improving hygiene and health care as a positive meaning rather than typical food products. Also, the Lactobacillus strain may also be used as forage for livestock, racehorses, and ornamental pets; and forages for animals such as pet foods.

### EXAMPLES

Hereinafter, the present invention will be described in detail with reference to the following Examples. However, it should be understood that the description presented herein is not intended to limit the scope of the present invention.

[Use Strains]

Strains listed in Table 1 were used as the Lactobacillus strain.

TABLE 1

| Strains names | Abbreviations |
|---|---|
| Lactobacillus plantarum SNK12 | SNK12 |
| (Accession No. NITE P-1445) | |
| Lactobacillus plantarum nF1 | nF1 |
| (Accession No. NITE P-1462) | |
| Lactobacillus plantarum KH3 | KH3 |
| (Accession No. NITE AP-1476) | |
| Enterococcus faecalis KH2 | KH2 |
| (Accession No. NITE P-1444) | |
| Lactobacillus brevis | Labre |
| (Accession No. FERM BP-4693) | |
| Lactococcus lactis sp cremoris | CF4 |
| (Accession No. FERM P-20848) | |

[Culturing of Lactobacillus Strain]

The culturing of the Lactobacillus strains was performed as follows, except that it was especially noted.

A “Difco Lactobacilli MRS broth” (trade name, Becton Dickinson & Company, Japan) was used as the culture medium. When it was assumed that the Lactobacillus strains were cultured at a starting strain concentration of 1×10⁶ to 1×10⁷ cfu/mL, the culturing conditions were set until the final strain concentration reaches 5×10⁹ to 5×10¹⁰ cfu/mL when the pH of the culture broth was set in a range of pH 6.0 to 6.5 at the start of this cultivation, and the Lactobacillus strains were then cultivated while adjusting pH. The cultivation was performed at temperatures suitable for the growth of the respective Lactobacillus strains, that is, a temperature of 32° C. on the Lactobacillus strains such as L. plantarum nF1, L. plantarum SNK12 and L. plantarum KH3, a temperature of 37° C. on the Lactobacillus strain such as E. faecalis KH2, a temperature of 31 to 32° C. on the Lactobacillus strain such as L. brevis FERP BP-4693, and a temperature of 30° C. on the Lactobacillus strain such as L. lactis sp. cremoris CF4. When the pH of the culture broth was adjusted, the cultivation was performed while the pH of the culture broth was adjusted with caustic soda at a setting of pH±0.1 using a pH stat.

[Post-Cultivation Process]

A post-cultivation process was performed as follows, except that it was especially noted.

90 mL of a culture broth was sterilized at 80° C. for 10 minutes in an autoclave, and centrifuged to collect the strain. Then, the collected strain was suspended in approximately 20 mL of a phosphate buffer until the strain concentration reached 1 to 4%. The resulting suspension was thoroughly dispersed using a Teflon homogenizer to prevent the strains from aggregating to each other. The strain suspension thus obtained was sterilized at 121° C. for 15 minutes, sonicated for 30 minutes to prevent the aggregation of the strains, and the solid content of the strain suspension was measured simultaneously or separately. Then, a IL-12 production induction test was performed by adjusting the strain suspension to a predetermined concentration, based on the measured solid content of the strain suspension.

[IL-12 Production Induction Test]

An in vitro culture system of mouse spleen cells was used in an IL-12 production induction test. Specifically, spleen cells were extracted from 8-week-old BALB/c mice, and a cell suspension (1.0×10⁶ cells/mL) was prepared in an RPMI1640 culture medium supplemented with 10% FBS, 10 μM 2-mercaptoethanol, 10 mM HEPES, 100 U/mL penicillin, and 100 μg/mL streptomycin according to a typical method. The Lactobacillus strain was added to the cell suspension until the final concentration reached 10 μg/mL when it was calculated as the dry matter of the Lactobacillus strain, and seeded to each well of a 96-well plate at an amount of 0.2 mL. The Lactobacillus strain was cultivated for 24 hours under conditions of a temperature of 37° C. and 5% CO₂, and a culture supernatant was recovered after the cultivation to measure an amount of IL-12 in the culture supernatant. Also, a kit “EMIL12” (trade name, commercially available from Thermo Scientific) was used for measurements, and the results were calculated as an average of 6 wells of an immunoplate.

### Experimental Example 1

L. plantarum nF1 was used as the Lactobacillus strain, and cultivated while adding an alkaline chemical (caustic soda) to a culture medium to adjust pH (hereinafter referred to as “neutralization culture”), and also cultivated without adjusting the pH (hereinafter referred to as “stationary culture”). Thereafter, the abilities to induce IL-12 production were compared for both of the Lactobacillus strains. Also, the abilities to induce IL-12 production were also compared between the Lactobacillus strains obtained from the stationary culture after the neutralization culture. The results are listed in Table 2.

TABLE 2

| | | | Ability to induce |
|---|---|---|---|
| Strain | | | IL-12 production |
| types | Culture conditions | Final pH | (pg/mL) |
| nF1 | 1. Stationary culture for 20 Hrs | 3.8 | 1,800 |
| | 2. Stationary culture for 0 Hrs | 6.1 | 50 |
| | after neutralization | | |
| | culture for 20 Hrs | | |
| | 3. Stationary culture for 2 Hrs | 5.5 | 700 |
| | after neutralization | | |
| | culture for 20 Hrs | | |
| | 4. Stationary culture for 4 Hrs | 5.0 | 1,300 |
| | after neutralization | | |
| | culture for 20 Hrs | | |
| | 5. Stationary culture for 6 Hrs | 4.9 | 1,400 |
| | after neutralization | | |
| | culture for 20 Hrs | | |
| | 6. Stationary culture for 16 Hrs | 7.8 | 1,550 |
| | after neutralization | | |
| | culture for 20 Hrs | | |

As listed in Table 2, the Lactobacillus strain, L. plantarum nF1 having an ability to induce IL-12 production, obtained from the stationary culture at 32° C. for 20 hours had an IL-12 production volume of 1,800 pg/mL. On the contrary, when the Lactobacillus strain was subjected to the neutralization culture in the same culture medium, the volume of the Lactobacillus strain increased by approximately three times, compared to when the Lactobacillus strain was subjected to the stationary culture, but the IL-12 production volume as the ability to induce IL-12 production was reduced to 50 pg/mL. On the other hand, when the neutralization culture was performed for 20 hours, a pH stat was turned off, and the stationary culture was performed, it was clearly revealed as a result that the ability to induce IL-12 production was enhanced as the stationary culture time was extended. Therefore, it was clearly revealed that a decrease in the ability to induce IL-12 production by means of the neutralization culture was recovered when the stationary culture was performed after the neutralization culture without controlling the pH.

### Experimental Example 2

The same test as in Experimental Example 1 was performed on the Lactobacillus strain, L. brevis FERP BP-4693. The results are listed in Table 3.

TABLE 3

| | | | Ability to induce |
|---|---|---|---|
| Strain | | Final | IL-12 production |
| types | Culture conditions | pH | (pg/mL) |
| Labre | 1. Stationary culture for 29 Hrs | 3.8 | 783 |
| | 2. Stationary culture for 0 Hrs | 6.1 | 152 |
| | after neutralization culture for | | |
| | 23 Hrs | | |
| | 3. Stationary culture (at 30° C.) | 4.7 | 439 |
| | for 60 Hrs after neutralization culture | | |
| | for 23 Hrs | | |
| | 4. Stationary culture (at 40° C.) | 4.5 | 897 |
| | for 60 Hrs after neutralization culture | | |
| | for 23 Hrs | | |

As shown in Table 3, the Lactobacillus strain, L. brevis FERP BP-4693 having an ability to induce IL-12 production, obtained from the stationary culture at 31° C. for 29 hours had an IL-12 production volume of 7.83 pg/mL. On the contrary, when the Lactobacillus strain was subjected to the neutralization culture in the same culture medium, the volume of the Lactobacillus strain increased by approximately three times, compared to when the Lactobacillus strain was subjected to the stationary culture, but the IL-12 production volume as the ability to induce IL-12 production was reduced to 152 pg/mL. On the other hand, when the neutralization culture was performed for 23 hours, a pH stat was turned off, and the stationary culture was performed, it was clearly revealed as a result that the ability to induce IL-12 production was enhanced as the stationary culture time was extended. Therefore, it was clearly revealed that a decrease in the ability to induce IL-12 production by means of the neutralization culture was recovered when the stationary culture was performed after the neutralization culture without controlling the pH, regardless of the type of the Lactobacillus strain.

### Experimental Example 3

From the results of Experimental Examples 1 and 2, it was revealed that the ability to induce IL-12 production was enhanced when the pH adjustment was suspended after the neutralization culture, and the stationary culture was then performed. Accordingly, it was tested whether the ability to induce IL-12 production was further enhanced by keeping the corresponding strains under severe conditions upon the stationary culture. Also, L. plantarum SNK12 was used as the Lactobacillus strain. The results are listed in Table 4.

TABLE 4

| | | | Ability to induce |
|---|---|---|---|
| Strain | | Final | IL-12 production |
| types | Culture conditions | pH | (pg/mL) |
| SNK12 | 1. Stationary culture for 24 Hrs | 3.7 | 1,050 |
| | 2. Stationary culture for 0 Hrs | 6.1 | 50 |
| | after neutralization culture for 20 Hrs | | |
| | 3. Stationary culture for 4 Hrs | 5.0 | 1,100 |
| | after neutralization culture for 20 Hrs | | |
| | 4. Stationary culture (at 40° C.) | 4.9 | 1,400 |
| | for 4 Hrs after neutralization culture | | |
| | for 20 Hrs | | |
| | 5. Stationary culture for 4 Hrs (in the | 5.1 | 1,250 |
| | presence of 1% NaCl) after | | |
| | neutralization culture for 20 Hrs | | |
| | 6. Stationary culture for 4 Hrs (in the | 5.2 | 1,750 |
| | presence of 2% NaCl) after | | |
| | neutralization culture for 20 Hrs | | |
| | 7. Stationary culture at 40° C. for | 5.1 | 1,850 |
| | 4 Hrs (in the presence of 2% NaCl) | | |
| | after neutralization culture for 20 Hrs | | |

As listed in Table 4, the Lactobacillus strain, L. plantarum SNK12 having an ability to induce IL-12 production, obtained from the stationary culture at 32° C. for 24 hours had an IL-12 production volume of 1,050 pg/mL. On the contrary, when the Lactobacillus strain was subjected to the neutralization culture for 20 hours in the same culture medium, the volume of the Lactobacillus strain increased by approximately three times, compared to when the Lactobacillus strain was subjected to the stationary culture, but the IL-12 production volume as the ability to induce IL-12 production was reduced to 50 pg/mL. On the other hand, when the neutralization culture was performed for 20 hours, a pH stat was turned off, and the stationary culture was performed for 4 hours, the IL-12 production volume increased to 1,100 pg/mL, which resulted in recovery of the ability to induce IL-12 production. Also, it was clearly revealed that the ability to induce IL-12 production was further enhanced when a saline solution was added to the culture medium upon the stationary culture, or the Lactobacillus strain, L. plantarum SNK12, was heated to a severe culture temperature of 40° C. to propagate the Lactobacillus strain. An effect of activating the ability to induce IL-12 production was shown to be highest when the stationary culture, the addition of saline solution, and the high temperature were combined.

From these results, it was clearly revealed that the ability of the Lactobacillus strain to induce IL-12 production was recovered or further enhanced when the corresponding strain was forcedly kept under the severe stress conditions for growth. Therefore, it was clearly revealed that a sufficient volume of the strain was obtained by applying stress after the neutralization culture, and the Lactobacillus strain having a high ability to induce IL-12 production was able to be prepared as well.

### Experimental Example 4

It is necessary to prevent the self-digestion of the Lactobacillus strain when the Lactobacillus strain is supplied as a preparation so as to secure the stability. As the process of sterilizing and collecting the strain after the cultivation is completed, two methods, that is, a method of removing the culture medium components by means of an MF membrane or centrifugation and sterilizing a concentrate of the Lactobacillus strain which is suspended in a medium such as water or a buffer, and a method of sterilizing the Lactobacillus strain in advance in a state in which a culture broth is left after the cultivation and collecting the Lactobacillus strain can be considered. Among these, it was examined which method was to be adopted. For the examination, the effects when the culture broth was sterilized at 80° C. for 10 minutes after the cultivation were compared to those when the Lactobacillus strain was collected by centrifugation, suspended in a phosphate buffer, and sterilized at the same conditions. The results are listed in Table 5.

TABLE 5

| | | Ability to induce |
|---|---|---|
| Strain | | IL-12 production |
| types | Culture and sterilization conditions | (pg/mL) |
| nF1 | Stationary culture for 6 Hrs, | 200 |
| | collection and sterilization after | |
| | neutralization culture for 27 Hrs | |
| | Stationary culture for 6 Hrs, | 1,000 |
| | sterilization and collection after | |
| | neutralization culture for 27 Hrs | |
| Labre | Stationary culture for 6 Hrs, | 120 |
| | collection and sterilization after | |
| | neutralization culture for 40 Hrs | |
| | Stationary culture for 6 Hrs, | 580 |
| | sterilization and collection after | |
| | neutralization culture for 40 Hrs | |
| SNK2 | Stationary culture for 6 Hrs, | 50 |
| | collection and sterilization after | |
| | neutralization culture for 20 Hrs | |
| | Stationary culture for 6 Hrs, | 1,100 |
| | sterilization and collection after | |
| | neutralization culture for 20 Hrs | |
| KH2 | Stationary culture for 6 Hrs, | 1,300 |
| | collection and sterilization after | |
| | neutralization culture for 17 Hrs | |
| | Stationary culture for 6 Hrs, | 2,100 |
| | sterilization and collection after | |
| | neutralization culture for 17 Hrs | |
| CF4 | Stationary culture for 6 Hrs, | 275 |
| | collection and sterilization after | |
| | neutralization culture for 18 Hrs | |
| | Stationary culture for 6 Hrs, | 738 |
| | sterilization and collection after | |
| | neutralization culture for 18 Hrs | |

As listed in Table 5, when any strain was sterilized in the culture broth after the cultivation, and then collected, the ability to induce IL-12 production was significantly higher, compared to when any strain was collected and then sterilized in a state in which the culture broth was removed. Also, when any strain was sterilized and collected, the Lactobacillus strain turned yellowish brown thickly, compared to when any strain was collected and sterilized. The reasons for these results being obtained were not clear, but one possibility was that any of physiological responses appeared in the Lactobacillus strain when the Lactobacillus strain was sterilized in the culture broth after the cultivation rather than when the Lactobacillus strain was collected and sterilized.

### Experimental Example 5

From the results obtained in Experimental Example 4, it was revealed that, when the Lactobacillus strain was sterilized in the culture broth after the cultivation, and then collected, the Lactobacillus strain had a higher ability to induce IL-12 production, compared to when the Lactobacillus strain was collected, and then sterilized in a state in which the culture broth was removed. Also, it was revealed that, when the strain was sterilized and collected, the Lactobacillus strain turned yellowish brown thickly, compared to when the strain was collected and sterilized. Accordingly, the relationship between the staining of the Lactobacillus strain and the ability to induce IL-12 production was further examined. Also, L. plantarum KH3 was used as the Lactobacillus strain, subjected to neutralization culture for 18 hours, followed by stationary culture for 6 hours. Thereafter, the Lactobacillus strain was sterilized by heating under the corresponding condition after the cultivation.

The staining of the Lactobacillus strain was subjected to colorimetry using a colorimeter (SQ-300H commercially available from Nippon Denshoku Industries Co., Ltd.), as follows.

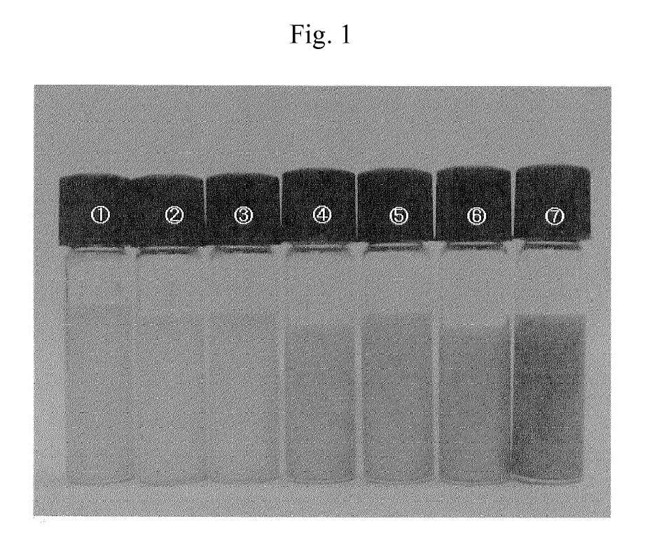

90 mL of a culture broth was taken after sterilization, and divided to 50 mL centrifuge tubes at a volume of 45 mL, and then centrifuged at 8,400 rpm for 5 minutes to collect the strain. The strain was suspended in a phosphate buffer, homogenized using a Teflon homogenizer, and then re-centrifuged to remove the culture medium components. The resulting precipitate was suspended in a phosphate buffer, and then homogenized using a Teflon homogenizer to finally prepare the Lactobacillus strain at a volume of 25 mL. The strain suspension having a solid content of 4% thus prepared was transferred to a quartz glass Petri dish (diameter: 35 mm), and the brightness L* value and chromaticity a* and b* values of the L*a*b* color system were measured in this situation using a colorimeter (SQ-300H commercially available from Nippon Denshoku Industries Co., Ltd.). Also, FIG. 1 shows an image of the strain suspension.

The results are summarized, and listed in Table 6.

TABLE 6

| | | | | | Ability |
|---|---|---|---|---|---|
| | | | | | to induce |
| | | | | | IL-12 |
| Strain | | | a* | | production |
| types | Sterilization conditions | L* value | value | b* value | (pg/mL) |
| KH3 | 1. Not | 66.18 | −6.23 | 15.61 | 622 |
| | sterilized | | | | |
| | 2. At 60° C. | 69.67 | −6.60 | 20.08 | 430 |
| | for 10 minutes | | | | |
| | 3. At 70° C. | 70.05 | −5.81 | 17.61 | 317 |
| | for 10 minutes | | | | |
| | 4. At 80° C. | 60.42 | −3.07 | 19.58 | 1,237 |
| | for 10 minutes | | | | |
| | 5. At 90° C. | 57.41 | −1.84 | 18.78 | 939 |
| | for 10 minutes | | | | |
| | 6. At 100° C. | 56.31 | −0.53 | 19.40 | 585 |
| | for 10 minutes | | | | |
| | 7. At 121° C. | n.d. | n.d. | n.d. | n.d. |
| | for 10 minutes | | | | |

n.d.: not determined

As listed in Table 6, the Lactobacillus strain has a higher ability to induce IL-12 production when the strain was sterilized under predetermined conditions after the cultivation, compared to when the strain was not sterilized. It was more preferred that the sterilization temperature was in a range of 80 to 100° C., and the improved ability to induce IL-12 production was very well correlated to a decrease in the brightness L* value and an increase in the chromaticity a* value of the strain.

1. A method for preparing a Lactobacillus strain having an ability to induce IL-12 production, the method comprising:
adding an alkaline chemical to a culture medium to adjust pH;
cultivating Lactobacillus sp. in the culture medium;
applying stress at a later stage of the cultivation; and
sterilizing the Lactobacillus sp.

2. The method of claim 1, wherein the stress is at least one selected from the group consisting of (a) cultivating the Lactobacillus sp. without adding the alkaline chemical; (b) cultivating the Lactobacillus sp. in a temperature range in which the propagation of the Lactobacillus sp. is suppressed; (c) cultivating the Lactobacillus sp. after a salt is added at a concentration of 1% by weight or more in a culture broth; and (d) cultivating the Lactobacillus sp. at pH 5 or less.

3. The method of claim 1, wherein the Lactobacillus sp. is sterilized in the culture broth after the cultivation, and then collected.

4. The method of claim 1, wherein the Lactobacillus sp. is isolated from a plant.

5. The method of claim 1, wherein the Lactobacillus sp. is a microorganism belonging to Lactobacillus plantarum, Lactococcus lactis sp. cremoris, Enterococcus faecalis, or Lactobacillus brevis.

6. The method of claim 1, wherein the sterilizing of the Lactobacillus sp. is performed at 80° C. or higher.

7. The method of claim 3, wherein the corresponding sterilization is performed so that when 5 g of strain concentrate having a solid content of 4 to 7% by weight prepared by Sterilizing the Lactobacillus after cultivation and Removing culture medium by centrifugation, membrane separation, or precipitation of the culture broth is added into a quartz glass Petri dish having a diameter of 35 mm,
the brightness L* value is less than or equal to 65 and a chromaticity a* value is greater than or equal to −4 as measured by a colorimetric color of the L*a*b* color system.

8. A Lactobacillus strain having an ability to induce IL-12 production, which is obtained by adding an alkaline chemical to a culture medium to adjust pH, cultivating Lactobacillus sp. in the culture medium,
applying stress at a later stage of the cultivation and sterilizing the Lactobacillus sp.

9. The Lactobacillus strain of claim 8, which is obtained by sterilizing the Lactobacillus sp. in a culture broth after the cultivation, followed by collecting the Lactobacillus sp.

10. The Lactobacillus strain of claim 8, which is obtained by sterilizing of the Lactobacillus sp. at 80° C. or higher.

11. The Lactobacillus strain of claim 9, which is obtained by performing the corresponding sterilization so that when 5 g of strain concentrate having a solid content of 4 to 7% by weight prepared by Sterilizing the Lactobacillus after cultivation and Removing culture medium by centrifugation, membrane separation, or precipitation of the culture broth is added into a quartz glass Petri dish having a diameter of 35 mm,
the brightness L* value is less than or equal to 65 and a chromaticity a* value is greater than or equal to −4 as measured by a colorimetric color of the L*a*b* color system.
